(19) United States
(12) Patent Application Publication
(10) Pub. No.: US 2020/0397325 A1
(43) Pub. Date: Dec. 24, 2020

(54) INTRATHORACIC PRESSURE SENSOR

(71) Applicants: Yoshitsugu YAMADA, Tokyo (JP); NIHON KOHDEN CORPORATION, Tokyo (JP)

(72) Inventor: Yoshitsugu YAMADA, Tokyo (JP)

(73) Assignees: Yoshitsugu YAMADA, Tokyo (JP); NIHON KOHDEN CORPORATION, Tokyo (JP)

(21) Appl. No.: 16/904,053

(22) Filed: Jun. 17, 2020

(30) Foreign Application Priority Data
Jun. 24, 2019 (JP) 2019-116272

(51) Int. Cl. A61B 5/03; A61B 5/00; A61B 5/087; A61M 16/04; A61M 16/00

(52) U.S. Cl. CPC A61B 5/036; A61B 5/6847; A61B 5/087; A61M 2016/0027; A61M 16/0003; A61B 2562/0247; A61M 16/0434

(57) ABSTRACT

An intrathoracic pressure sensor includes: a first tube that is adapted to be inserted into a trachea of a patient to define a first gas passage configured to communicate with the trachea; a first cuff disposed in a portion of the first tube which is to be placed in the trachea, the first cuff being inflatable and deflatable; a second tube that defines a second gas passage configured to communicate with an interior of the first cuff; and a transducer configured to output a signal corresponding to a pressure in the second gas passage.

### CROSS-REFERENCE TO RELATED APPLICATIONS

This application is based upon and claims the benefit of priority from prior Japanese patent application No. 2019-116272, filed on Jun. 24, 2019, the entire contents of which are incorporated herein by reference.

The presently disclosed subject matter relates to a sensor that detects the intrathoracic pressure of a patient.

### BACKGROUND

The amount of change of the intrathoracic pressure due to respiration can be used as an index that is effective in diagnosis of a respiratory disease and evaluation of the pulmonary function. It is difficult to detect the intrathoracic pressure. Usually, a detection of the esophageal pressure is used as a substitute for detection of the intrathoracic pressure. U.S. Pat. No. 4,214,593 discloses a device in which a balloon catheter is inserted into the esophagus of a patient to detect the intrathoracic pressure of the patient.

The presently disclosed subject matter provides with a sensor for detecting the intrathoracic pressure.

### SUMMARY

According to an aspect of the presently disclosed subject matter, there is provided an intrathoracic pressure sensor comprising: a first tube that is adapted to be inserted into a trachea of a patient to define a first gas passage which is configured to communicate with the trachea; a first cuff that is disposed in a portion of the first tube which is to be placed in the trachea, the first cuff being inflatable and deflatable; a second tube that defines a second gas passage which is configured to communicate with an interior of the first cuff; and a transducer that is configured to output a signal corresponding to a pressure in the second gas passage.

### BRIEF DESCRIPTION OF DRAWINGS

FIG. 1 exemplarily illustrates the configuration of an intrathoracic pressure sensor of an embodiment.

FIGS. 2A and 2B exemplarily illustrate a method of using the intrathoracic pressure sensor of FIG. 1.

FIG. 3 illustrates another example of the configuration of the intrathoracic pressure sensor of FIG. 1.

### DESCRIPTION OF EMBODIMENTS

Hereinafter, an embodiment will be described in detail with reference to the accompanying drawings. In the drawings, in order to make components of a described object recognizable, the scales of the components are adequately changed.

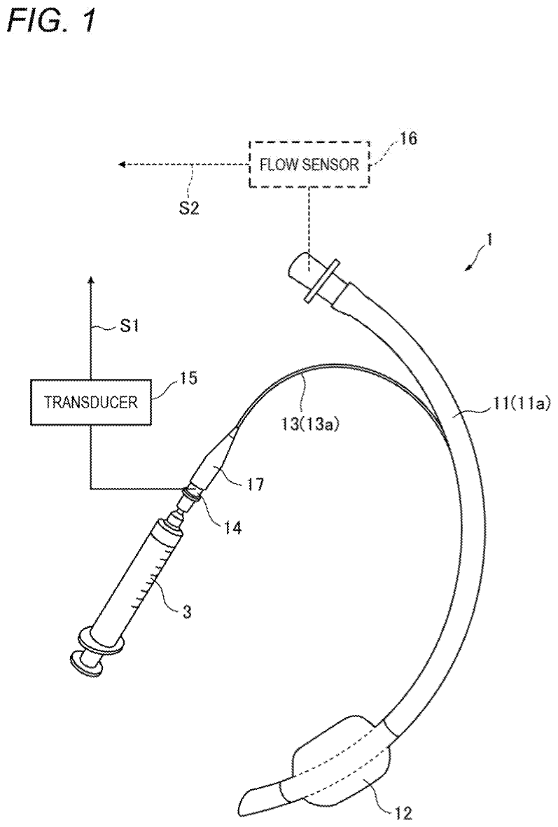

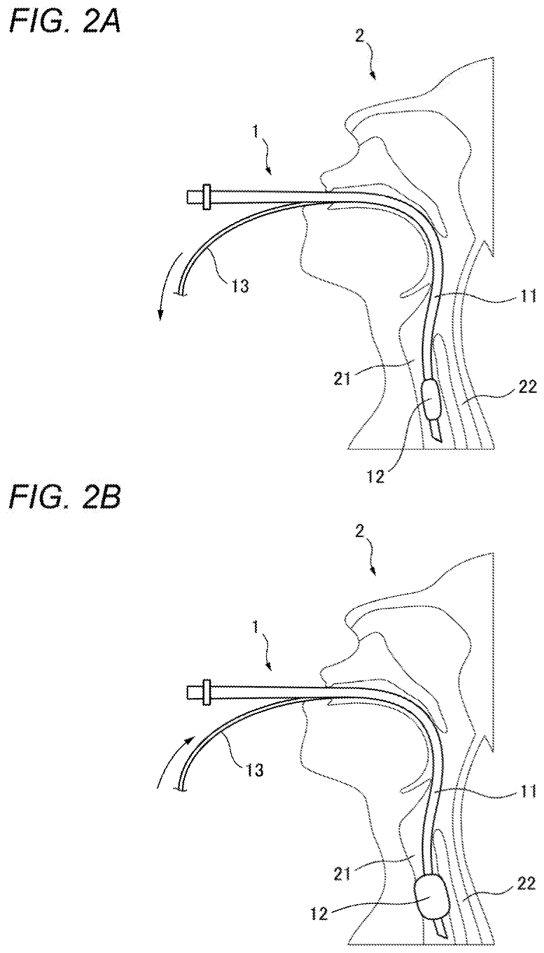

FIG. 1 exemplarily illustrates the configuration of an intrathoracic pressure sensor 1 of an embodiment. The intrathoracic pressure sensor 1 may include a first tube 11. As illustrated in FIG. 2A, the first tube 11 is configured so as to be inserted into a trachea 21 of a patient 2. The first tube 11 is formed by a material containing, for example, polyvinyl chloride, and has a flexibility that allows the first tube to bend according to the shape of the trachea 21.

The first tube 11 defines a first gas passage 11a. When the first tube 11 is inserted into the trachea 21, the first gas passage 11a communicates with the trachea 21. The first gas passage 11a can be used as a passage for a respiratory gas during respiratory support with a respirator, or a passage for supplying an anesthesia gas during surgery.

As illustrated in FIG. 1, the intrathoracic pressure sensor 1 may include a cuff 12. The cuff 12 is an inflatable and deflatable bag that is disposed along the outer periphery in the position of the first tube 11 which is to be placed in the trachea 21. The cuff 12 is formed by a material containing polyvinyl chloride or polyurethane.

The intrathoracic pressure sensor 1 may include a second tube 13. The second tube 13 defines a second gas passage 13a. The second gas passage 13a communicates with the interior of the bag forming the cuff 12. The second gas passage 13a does not communicate with the first gas passage 11a.

The intrathoracic pressure sensor 1 may include a check valve 14. The check valve 14 is placed in an end portion of the second gas passage 13a. The check valve 14 is configured so as to, when a syringe 3 is connected thereto, cause the second gas passage 13a to communicate with the interior of the syringe 3, and, when the syringe 3 is detached, seal the second gas passage 13a from the ambient air.

The intrathoracic pressure sensor 1 may include a transducer 15. The transducer 15 outputs a signal S1 corresponding to the pressure in the second gas passage 13a. Namely, a pressure detecting section of the transducer 15 communicates with the second gas passage 13a. For example, the transducer 15 is placed on the side of the check valve 14 that faces the second gas passage 13a.

A method of using the intrathoracic pressure sensor 1 will be described. First, the syringe 3 is connected to the check valve 14 to cause the interior of the syringe 3 to communicate with the second gas passage 13a, and then the air in the second gas passage 13a is sucked by the syringe 3. This causes also the air in the cuff 12 that communicates with the second gas passage 13a, to be sucked, thereby producing a state where the cuff 12 is deflated.

In this state, as illustrated in FIG. 2A, the first tube 11 is inserted into the trachea 21 of the patient 2. When the tube is inserted to an adequate position, the syringe 3 is operated to inject the air into the second gas passage 13a. Therefore, the air is injected into the cuff 12 that communicates with the second gas passage 13a, and, as illustrated in FIG. 2B, the cuff 12 is inflated. The outer surface of the cuff 12 is contacted with the inner wall of the trachea 21, and the trachea 21 is obstructed. The inner and outer sides of the trachea 21 of the patient 2 are caused to communicate with each other by the first gas passage 11a of the first tube 11.

A change of the intrathoracic pressure due to the respiration of the patient 2 is reflected in a change of the internal pressure of the cuff 12 that is in contact with the inner wall of the trachea 21. The pressure change in the cuff 12 is reflected in that in the second gas passage 13a. Therefore, the signal S1 corresponding to the pressure in the second gas passage 13a is output from the transducer 15, and, when the signal S1 is monitored, it is possible to detect the intrathoracic pressure of the patient 2.

The first tube 11, the cuff 12, and the second tube 13 are used also as an intratracheal tube that is used in intratracheal intubation. Also during a time period when the intrathoracic pressure of the patient 2 is detected by the transducer 15, an operation on the patient 2 such as respiratory support through the first gas passage 11a can be performed. Therefore, it is not necessary to insert a balloon catheter into the esophagus 22 of the patient 2 in order to detect the esophageal pressure that functions only to provide an alternative value for the intrathoracic pressure. Consequently, a space is formed through which another necessary probe or the like (such as a gastric tube, a transesophageal echo probe, or an esophageal temperature probe) can be inserted into the esophagus 22, and moreover detection of the esophageal pressure by a balloon catheter inserted into the esophagus 22 is not blocked by such a probe or the like. As a result, it is possible to provide an alternative technique in which the intrathoracic pressure can be detected without disturbing respiratory support, detection of the esophageal pressure, etc.

As illustrated in FIG. 1, the intrathoracic pressure sensor 1 may include a flow sensor 16. The flow sensor 16 communicates with the first gas passage 11a of the first tube 11, and outputs a signal S2 corresponding to the intraoral pressure of the patient 2.

According to the configuration, it is possible to detect the transpulmonary pressure of the patient 2. It is proposed that the transpulmonary pressure can be used as an index that is important in setting of a respirator which can avoid damage of the lung tissue. On the other hand, it is known that the intraoral pressure is expressed as the sum of the intrathoracic pressure and the transpulmonary pressure. When the difference between the intraoral pressure detected by the flow sensor 16 and the intrathoracic pressure detected by the transducer 15 is calculated, therefore, it is possible to acquire the amount of change of the transpulmonary pressure of the patient 2.

As illustrated in FIG. 1, the intrathoracic pressure sensor 1 may include a pilot cuff 17. The pilot cuff 17 communicates with the second gas passage 13a and the check valve 14. The pilot cuff 17 is an inflatable and deflatable bag that is smaller in capacity than the cuff 12. The pilot cuff 17 is an example of the second cuff.

Since the pilot cuff 17 communicates with the cuff 12 through the second gas passage 13a, the internal pressure of the pilot cuff 17 reflects that of the cuff 12. When the degree of inflation of the pilot cuff 17 is checked through at least one of the visual sense and the tactile sense, therefore, the condition of the cuff 12 can be known from the outside of the body of the patient 2. In the case where anesthesia gas flows through the first gas passage 11a, moreover, the rising of the internal pressure of the cuff 12 due to diffusion of the anesthesia gas can be suppressed to a predetermined level or lower. An example of the predetermined level is the maximum arterial capillary perfusion pressure.

The above-described embodiment is a mere example for facilitating understanding of the presently disclosed subject matter. The configuration of the embodiment may be adequately changed or improved without departing from the spirit of the presently disclosed subject matter.

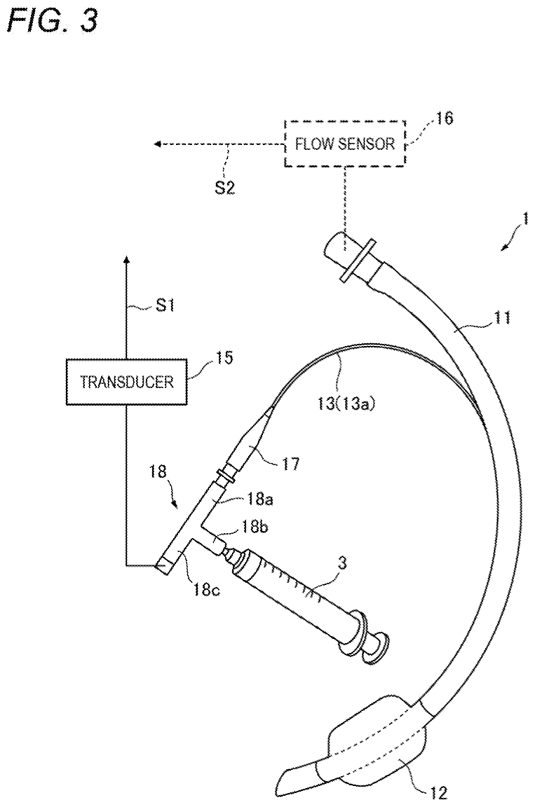

As illustrated in FIG. 3, the check valve 14 in the configuration example illustrated in FIG. 1 may be replaced with a three-way valve 18. The three-way valve 18 may include a first path 18a, a second path 18b, and a third path 18c. The first path 18a is connected to the second tube 13 so as to communicate with the second gas passage 13a. The second path 18b is connected to the syringe 3. The third path 18c is connected to the transducer 15. The three-way valve 18 is configured so that either one of the second path 18b and the third path 18c can be connected to the first path 18a.

When the intrathoracic pressure sensor 1 is to be inserted into the trachea 21 or removed from the trachea 21, the three-way valve 18 is operated so that the second path 18b communicates with the first path 18a. This enables the cuff 12 to be inflated or deflated by an operation of the syringe 3.

The inflation of the cuff 12 causes the position of the intrathoracic pressure sensor 1 in the trachea 21 to be determined, the three-way valve 18 is operated so that the third path 18c communicates with the first path 18a. This enables the pressure in the second gas passage 13a to be detected through the transducer 15.

When the second path 18b of the three-way valve 18 is opened to the atmosphere, the zero-point calibration in the detection of the pressure in the second gas passage 13a can be easily performed. Namely, a reference value for enhancing the measurement accuracy of the intrathoracic pressure can be readily determined. When the reference value is used, it becomes easy to continuously monitor a result of the measurement of the intrathoracic pressure by a patient monitor or the like.

What is claimed is:

1. An intrathoracic pressure sensor comprising:
a first tube adapted to be inserted into a trachea of a patient to define a first gas passage configured to communicate with the trachea;
a first cuff disposed in a portion of the first tube which is to be placed in the trachea, the first cuff being inflatable and deflatable;
a second tube that defines a second gas passage which is configured to communicate with an interior of the first cuff; and
a transducer configured to output a signal corresponding to a pressure in the second gas passage.

2. The intrathoracic pressure sensor according to claim 1, further comprising:
a flow sensor configured to communicate with the first gas passage, and that is configured to output a signal corresponding to an intraoral pressure of the patient.

3. The intrathoracic pressure sensor according to claim 1, further comprising:
a second cuff configured to communicate with the second gas passage, that is smaller in capacity than the first cuff, and that is inflatable and deflatable.

4. The intrathoracic pressure sensor according to claim 1, wherein
the first cuff is formed by a material containing polyvinyl chloride or polyurethane.
